(19) United States
(12) Patent Application Publication
(10) Pub. No.: US 2024/0298888 A1
(43) Pub. Date: Sep. 12, 2024

(54) CAMERA HEAD, ENDOSCOPE SYSTEM, AND METHOD OF MANUFACTURING CAMERA HEAD

(71) Applicant: Sony Olympus Medical Solutions Inc., Tokyo (JP)

(72) Inventors: Yasuhiro OKABE, Tokyo (JP); Atsuomi OHNO, Tokyo (JP); Shota HOKE, Tokyo (JP)

(73) Assignee: Sony Olympus Medical Solutions Inc., Tokyo (JP)

(21) Appl. No.: 18/433,733

(22) Filed: Feb. 6, 2024

(30) Foreign Application Priority Data
Mar. 6, 2023 (JP) 2023-033943

(51) Int. Cl. A61B 1/04; A61B 1/00

(52) U.S. Cl. CPC A61B 1/042; A61B 1/0011; A61B 1/00112

(57) ABSTRACT

A camera head includes: an exterior casing configured to be connected to an insertion unit, the insertion unit being inserted into a subject and configured to take in a subject image from the subject; an imaging unit provided in the exterior casing and configured to capture the subject image; and a heat sink provided in the exterior casing and configured to transfer heat generated in the imaging unit to the exterior casing.

### CROSS-REFERENCE TO RELATED APPLICATION

This application claims priority from Japanese Application No. 2023-033943, filed on Mar. 6, 2023, the contents of which are incorporated by reference herein in its entirety.

### BACKGROUND

The present disclosure relates to a camera head, an endoscope system and a method of manufacturing a camera head.

In the related art, there is known an endoscope system that captures an image of an inside of a subject such as a human (inside of a living body) using an imaging element and observes the inside of the living body (See, for example, WO 2018/116533 A).

An endoscope system described in WO 2018/116533 A includes an endoscope that is inserted into the living body and captures a subject image from the inside of the living body, and a camera head that is detachably connected to the endoscope and includes an imaging unit that captures the subject image captured by the endoscope.

### SUMMARY

Meanwhile, when an imaging unit is driven, heat is generated from the imaging unit. Therefore, a structure for efficiently dissipating heat generated from the imaging unit to the outside of the exterior casing housing the imaging unit is necessary. There is a case where the heat dissipation from the imaging unit is insufficient in the structure of the camera head described in WO 2018/116533 A depending on the design of the imaging unit, the exterior casing, and the like, and thus, a technique capable of improving the heat dissipation from the imaging unit is desired.

According to one aspect of the present disclosure, there is provided a camera head including: an exterior casing configured to be connected to an insertion unit, the insertion unit being inserted into a subject and configured to take in a subject image from the subject; an imaging unit provided in the exterior casing and configured to capture the subject image; and a heat sink provided in the exterior casing and configured to transfer heat generated in the imaging unit to the exterior casing.

According to another aspect of the present disclosure, there is provided an endoscope system including: an insertion unit configured to be inserted into a subject and take in a subject image from the subject; a camera head configured to be connected to the insertion unit and capture the subject image; and a control device configured to process an image captured by the camera head, wherein the camera head includes an exterior casing configured to be connected to the insertion unit, an imaging unit provided in the exterior casing and configured to capture the subject image, and a heat sink provided in the exterior casing and configured to transfer heat generated in the imaging unit to the exterior casing.

According to still another aspect of the present disclosure, there is provided a method of manufacturing a camera head including: an exterior casing configured to be connected to an insertion unit, the insertion unit being inserted into a subject and configured to take in a subject image from the subject; an imaging unit provided in the exterior casing and configured to capture the subject image; and a heat sink provided in the exterior casing and configured to transfer heat generated in the imaging unit to the exterior casing, the method including: a first assembling step of assembling the imaging unit and the heat sink to form an image-capturing unit; and a second assembling step of inserting the image-capturing unit into the exterior casing and assembling the image-capturing unit and the exterior casing after the first assembling step.

### BRIEF DESCRIPTION OF THE DRAWINGS

FIG. 1 is a view illustrating a configuration of an endoscope system according to an embodiment; FIG. 2 is a view describing a configuration of a camera head;

FIG. 3 is a view describing the configuration of the camera head;

FIG. 4 is a view describing the configuration of the camera head;

FIG. 5 is a view describing the configuration of the camera head;

FIG. 6 is a view describing the configuration of the camera head;

FIG. 7 is a view describing the configuration of the camera head;

FIG. 8 is a view describing the configuration of the camera head;

FIG. 9A is a view describing a first modification of the embodiment;

FIG. 9B is a view describing the first modification of the embodiment;

FIG. 10 is a view describing a second modification of the embodiment;

FIG. 11 is a view describing the second modification of the embodiment;

FIG. 12 is a view describing the second modification of the embodiment;

FIG. 13 is a view describing a third modification of the embodiment; and

FIG. 14 is a view describing the third modification of the embodiment.

### DETAILED DESCRIPTION

Hereinafter, modes (embodiments) for carrying out the present disclosure will be described with reference to the drawings. Note that the present disclosure is not limited by the embodiments described below. Furthermore, in the description of the drawings, the same portions are denoted by the same reference numerals.

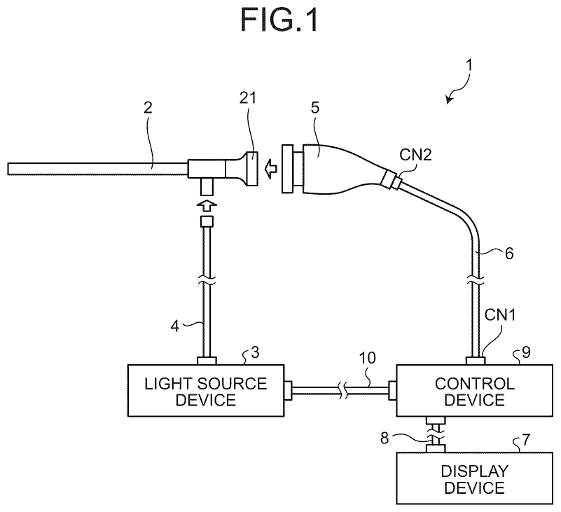

FIG. 1 is a view illustrating a schematic configuration of an endoscope system 1 according to an embodiment.

The endoscope system 1 is a system that is used in a medical field and observes the inside of a subject (inside of living body). As illustrated in FIG. 1, the endoscope system 1 includes an insertion unit 2, a light source device 3, a light guide 4, a camera head 5, a first transmission cable 6, a display device 7, a second transmission cable 8, a control device 9, and a third transmission cable 10.

In the present embodiment, the insertion unit 2 includes a rigid endoscope. That is, the insertion unit 2 has an elongated shape that is entirely rigid or partially soft and partially rigid, and is inserted into the living body. An optical system that includes one or a plurality of lenses and collects light (subject image) from the inside of the living body is provided in the insertion unit 2.

The light source device 3 is connected to one end of the light guide 4, and supplies light for illuminating the inside of the living body to the one end of the light guide 4 under the control of the control device 9.

In the present embodiment, the light source device 3 is configured separately from the control device 9, but the present disclosure is not limited thereto, and a configuration provided inside the control device 9 may be adopted.

One end of the light guide 4 is detachably connected to the light source device 3, and the other end is detachably connected to the insertion unit 2. Then, the light guide 4 transfers the light supplied from the light source device 3 from one end to the other end, and supplies the light to the insertion unit 2. The light supplied to the insertion unit 2 is emitted from the distal end of the insertion unit 2 and emitted into the living body. The light (subject image) emitted into the living body and reflected in the living body is collected by the optical system in the insertion unit 2.

The camera head 5 is detachably connected to the proximal end (eyepiece unit 21 (FIG. 1)) of the insertion unit 2. Then, under the control of the control device 9, the camera head 5 captures the subject image condensed by the insertion unit 2, and outputs an image signal (RAW signal) by the imaging. The image signal is, for example, an image signal of 4K or more.

Note that a detailed configuration of the camera head 5 will be described later.

One end of the first transmission cable 6 is detachably connected to the control device 9 via a connector CN1 (FIG. 1), and the other end is detachably connected to the camera head 5 via a connector CN2 (FIG. 1). Then, the first transmission cable 6 transmits an image signal and the like output from the camera head 5 to the control device 9, and transmits a control signal, a synchronization signal, a clock, power, and the like output from the control device 9 to the camera head 5.

In the transmission of the image signal and the like from the camera head 5 to the control device 9 via the first transmission cable 6, the image signal and the like may be transmitted as an optical signal or may be transmitted as an electric signal. The same applies to transmission of a control signal, a synchronization signal, and a clock from the control device 9 to the camera head 5 via the first transmission cable 6.

The display device 7 includes a display using liquid crystal, organic electro luminescence (EL), or the like, and displays an observation image based on a video signal from the control device 9 under the control of the control device 9.

One end of the second transmission cable 8 is detachably connected to the display device 7, and the other end is detachably connected to the control device 9. Then, the second transmission cable 8 transmits the video signal processed by the control device 9 to the display device 7.

The control device 9 includes a central processing unit (CPU) and the like, and integrally controls operations of the light source device 3, the camera head 5, and the display device 7.

Specifically, the control device 9 generates a video signal by performing various processes on the image signal acquired from the camera head 5 via the first transmission cable 6, and outputs the video signal to the display device 7 via the second transmission cable 8. Then, the display device 7 displays an image based on the video signal. In addition, the control device 9 outputs a control signal or the like to the camera head 5 and the light source device 3 via the first and third transmission cables 6 and 10.

One end of the third transmission cable 10 is detachably connected to the light source device 3, and the other end is detachably connected to the control device 9. Then, the third transmission cable 10 transmits the control signal from the control device 9 to the light source device 3.

Next, a configuration of the camera head 5 will be described.

Note that, in the following description, an X axis, a Y axis, and a Z axis orthogonal to each other are used to describe the configuration of the camera head 5. The Z axis is an axis along an up-down direction in FIG. 2. The X axis is an axis along a left-right direction in FIG. 2. The Y axis is an axis orthogonal to a paper surface of FIG. 2. In the following description, a side to which the proximal end (eyepiece unit 21) of the insertion unit 2 is connected is referred to as a distal end side Ar1, and a side to which the first transmission cable 6 is connected is referred to as a proximal end side Ar2.

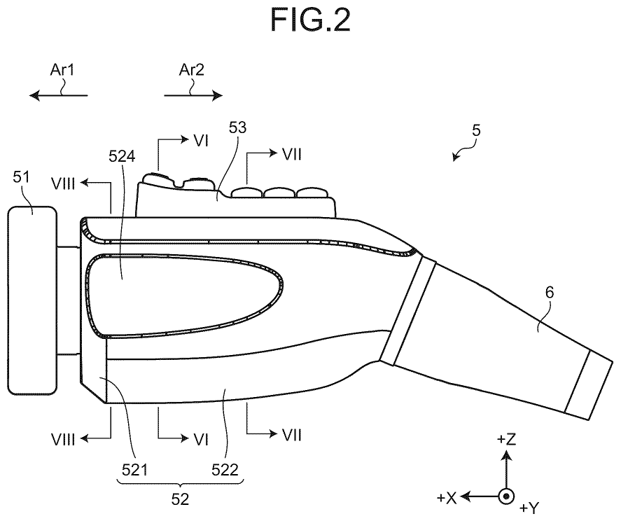

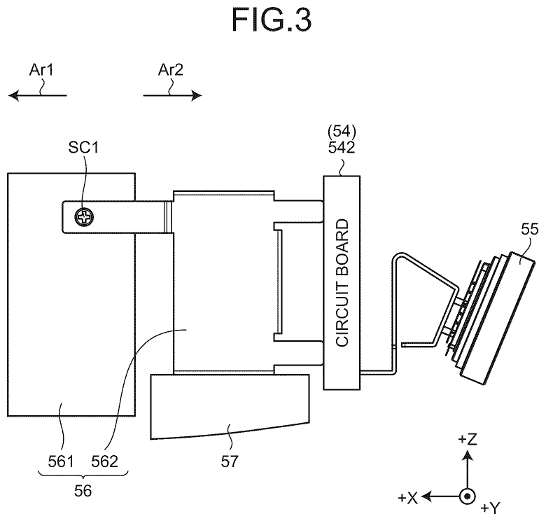

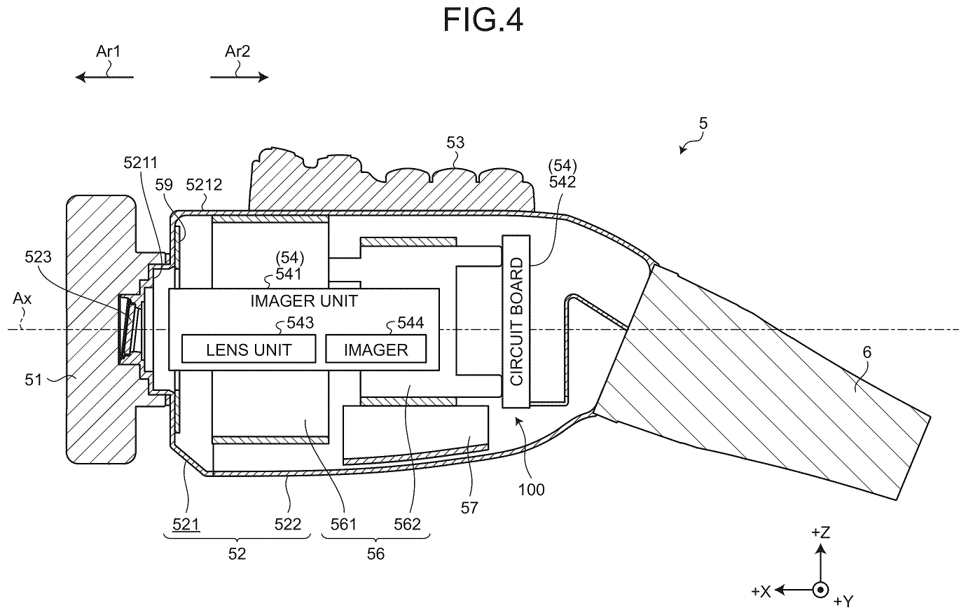

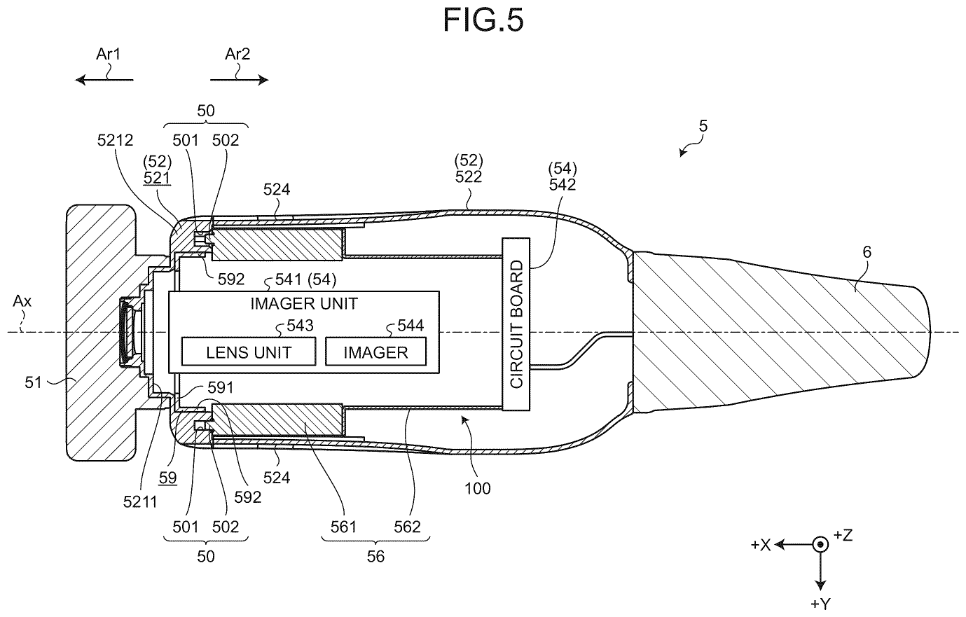

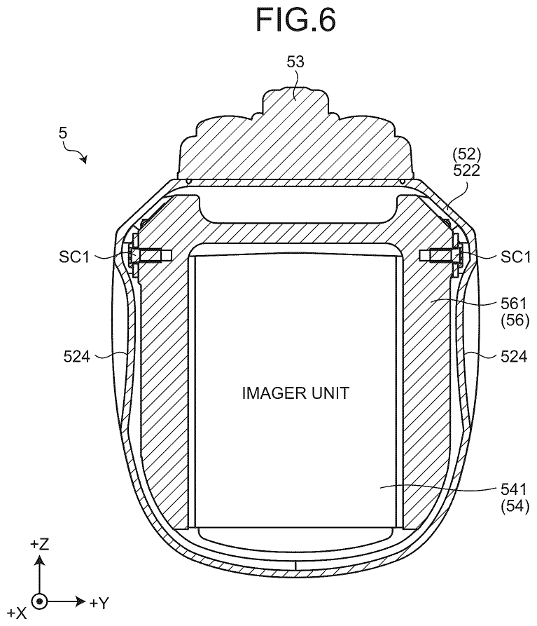

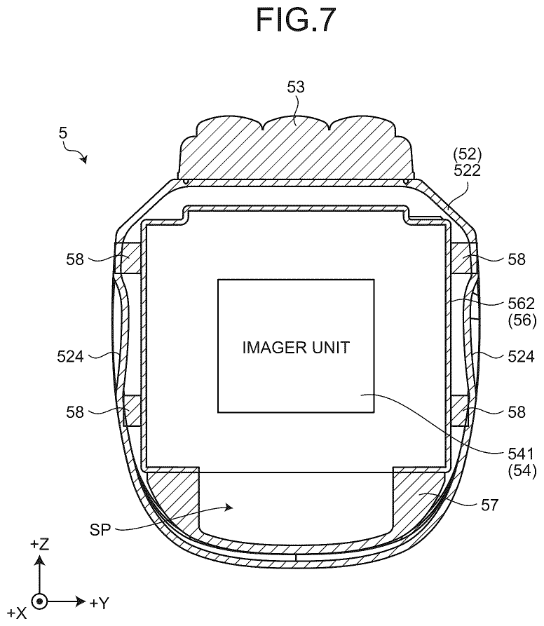

FIGS. 2 to 8 are views describing the configuration of the camera head 5. Specifically, FIG. 2 is a view illustrating an appearance of the camera head 5. FIG. 3 is a view illustrating an internal configuration of an exterior casing 52 in the camera head 5. Note that, in FIG. 3, illustration of the imaging unit 54 is omitted for convenience of description. FIG. 4 is a cross-sectional view of the camera head 5 taken along an XZ plane including an optical axis Ax of a lens unit 543. FIG. 5 is a cross-sectional view of the camera head 5 taken along the XY plane including the optical axis Ax. FIG. 6 is a cross-sectional view taken along line VI-VI of FIG. 2. FIG. 7 is a cross-sectional view taken along line VII-VII of FIG. 2. FIG. 8 is a cross-sectional view taken along line VIII-VIII in FIG. 2.

As illustrated in FIGS. 2 to 8, the camera head 5 includes a coupler unit 51 (FIGS. 2, 4, and 5), an exterior casing 52 (FIGS. 2 and 4 to 8), an operating unit 53 (FIGS. 2, 4, 6, and 7), an imaging unit 54 (FIGS. 4 to 8), a hermetic connector 55 (FIG. 3), a support member 56 (FIGS. 3 to 8), a heat sink 57 (FIGS. 3, 4, and 7), a heat transfer member 58 (FIG. 7), and a thermally conductive sheet 59 (FIGS. 5 and 8).

The coupler unit 51 is formed of a substantially cylindrical member, and the proximal end (eyepiece unit 21) of the insertion unit 2 is detachably connected to the coupler unit. The coupler unit 51 is connected to an end portion of the exterior casing 52 on the distal end side Ar1.

The exterior casing 52 is a portion to which the insertion unit 2 is connected and which is gripped by a user such as a doctor. In the present embodiment, as illustrated in FIG. 2 and FIGS. 4 to 8, the exterior casing 52 includes two bodies of a front casing 521 and a rear casing 522.

The front casing 521 is made of aluminum, an aluminum alloy, stainless steel, titanium, a titanium alloy, or the like, and has a tubular shape. More specifically, as illustrated in FIG. 4 or 5, the front casing 521 includes a distal end portion 5211 having a shape that gradually narrows an aperture area toward the distal end side Ar1, and a tubular proximal end portion 5212 extending from an end portion of the proximal end side Ar2 of the distal end portion 5211 toward the proximal end side Ar2. Then, an optical element 523 (FIGS. 4 and 5) is fixed to an end portion of the distal end portion 5211 on the distal end side Ar1 by, for example, brazing. The optical element 523 is made of, for example, flat sapphire glass. That is, the optical element 523 hermetically seals the aperture of the front casing 521 on the distal end side Ar1.

The rear casing 522 is made of aluminum, an aluminum alloy, stainless steel, titanium, a titanium alloy, or the like, and has a tubular shape. Then, the rear casing 522 is fixed to the front casing 521 by being welded in a state where an end portion on the distal end side Ar1 abuts on an end portion of the front casing 521 (proximal end portion 5212) on the proximal end side Ar2. That is, the front casing 521 and the rear casing 522 are hermetically sealed.

On the outer surface of the exterior casing 52 described above, as illustrated in FIG. 2, recesses 524 which are recessed toward the inside and are gripped by a user such as a doctor are formed on outer surfaces on both sides in the Y axis direction. The recess 524 extends from the distal end side Ar1 toward the proximal end side Ar2 across a boundary portion between the front casing 521 and the rear casing 522.

As illustrated in FIGS. 2, 4, 6, and 7, the operating unit 53 is fixed to the outer surface of a rear casing 512 by welding and receives an operation by a user such as a doctor. Then, the operating unit 53 outputs an operation signal corresponding to an operation by a user such as a doctor. Although not specifically illustrated, the operating unit 53 is electrically connected to an internal substrate (not illustrated) provided in the rear casing 512 via a hermetic connector (not illustrated) provided in the rear casing 512. That is, the operation signal from the operating unit 53 is output to the internal substrate via the hermetic connector.

The imaging unit 54 is disposed in the exterior casing 52, and captures a subject image collected by the insertion unit 2 and passing through the optical element 523. As illustrated in FIGS. 4 to 8, the imaging unit 54 includes an imager unit 541 and a circuit board 542.

As illustrated in FIGS. 4 to 8, the imager unit 541 includes a lens unit 543 and an imager 544.

The lens unit 543 is disposed on the proximal end side Ar2 with respect to the optical element 523, is condensed by the insertion unit 2, and forms the subject image through the optical element 523 on an imaging surface of the imager 544.

The imager 544 includes a charge coupled device (CCD), a complementary metal oxide semiconductor (CMOS), or the like that is disposed closer to the proximal end side Ar2 than the lens unit 543, receives the subject image focused by the insertion unit 2 and formed by the lens unit 543 via the optical element 523, and converts the subject image into an electric signal. Then, the imager 544 captures an image inside the living body under the control of the control device 9.

Although not specifically illustrated, the lens unit 543 and the imager 544 described above are fixed to each other by a holding member and unitized.

The circuit board 542 is disposed on the proximal end side Ar2 of the imager 544 and is electrically connected to the imager 544. Then, the circuit board 542 drives the imager 544 under the control of the control device 9. In addition, the circuit board 542 outputs the image signal obtained by imaging by the imager 544.

The hermetic connector 55 is fixed to an end portion of the rear casing 522 on the proximal end side Ar2 by welding, and airtightly seals an aperture on the proximal end side Ar2 of the rear casing 522. In addition, the hermetic connector 55 is electrically connected to the internal substrate and the circuit board 542 described above. Further, the connector CN2 is detachably connected to the hermetic connector 55. That is, the operation signal from the operating unit 53 is output to the control device 9 via the internal substrate, the hermetic connector 55, and the first transmission cable 6 described above. The image signal output from the circuit board 542 is output to the control device 9 via the hermetic connector 55 and the first transmission cable 6. Furthermore, the control signal and the like output from the control device 9 are output to the circuit board 542 and the like via the first transmission cable 6 and the hermetic connector 55.

The support member 56 is disposed in the exterior casing 52 and supports the imaging unit 54. As illustrated in FIGS. 3 to 8, the support member 56 includes a first support member 561 and a second support member 562.

The first support member 561 is made of a thermally conductive material such as metal and has a substantially tubular shape. The first support member 561 supports the imager unit 541. In a state where the first support member 561 supports the imager unit 541, the lens unit 543 is positioned inside the first support member 561, and a part thereof protrudes from the aperture of the first support member 561 on the distal end side Ar1 toward the distal end side Ar1. In addition, the imager 544 is located on the proximal end side Ar2 with respect to the first support member 561.

A fastening structure 50 (FIGS. 5 and 8) for fastening the support member 56 and the exterior casing 52 is provided between the first support member 561 and the exterior casing 52.

The fastening structure 50 corresponds to a fastening unit according to the present disclosure. As illustrated in FIGS. 5 and 8, the fastening structure 50 includes a fastening hole 501 and a fastening protrusion 502.

As illustrated in FIG. 5, the fastening hole 501 is provided on the end surface of the front casing 521 (proximal end portion 5212) on the proximal end side Ar2, and is a hole recessed from the end surface toward the distal end side Ar1. In the present embodiment, as illustrated in FIG. 8, five fastening holes 501 are provided.

As illustrated in FIG. 5, the fastening protrusion 502 is a protrusion provided on the end surface of the first support member 561 on the distal end side Ar1 and protruding from the end surface toward the distal end side Ar1. In the present embodiment, as illustrated in FIG. 8, the number of fastening protrusions 502 is the same as the number of fastening holes 501. The fastening protrusion 502 is fitted into the fastening hole 501. With such a configuration, the support member 56 and the exterior casing 52 are fastened to each other.

That is, the fastening structure 50 is located at a boundary portion between the front casing 521 and the rear casing 522. The fastening structure 50 is disposed opposite to the back surface of the recess 524.

The second support member 562 is made of a thermally conductive material such as metal and has a substantially tubular shape. The second support member 562 supports the circuit board 542. In addition, the imager 544 supported by the first support member 561 is positioned in the second support member 562. Further, the second support member 562 is located on the proximal end side Ar2 with respect to the first support member 561, and is connected to the first support member 561 by a screw SC1 (FIGS. 3 and 6). That is, the second support member 562 is thermally connected to the first support member 561.

The heat sink 57 is made of a thermally conductive material such as metal, and is disposed at a position facing the operating unit 53 with the second support member 562 (imaging unit 54) interposed therebetween in the exterior casing 52 as illustrated in FIGS. 3, 4, and 7. The heat sink 57 is directly connected to the second support member 562. That is, the heat sink 57 is thermally connected to the second support member 562. The heat sink 57 is not directly connected to the first support member 561.

As illustrated in FIG. 7, the heat sink 57 has a U-shaped cross section in which the second support member 562 side is opened. That is, a space SP is formed between the heat sink 57 and the second support member 562.

In the heat sink 57, the outer surface facing the inner surface of the rear casing 522 has a shape following the inner surface as illustrated in FIG. 7.

When a cross-sectional area obtained by cutting the space formed by the support member 56 and the outer surface of the heat sink 57 described above along the YZ plane is x, there is a location where a distance between the support member 56 or the outer surface of the heat sink 57 and the inner surface of the exterior casing 52 is equal to or more than a value obtained by multiplying a square root of the x by 0.0025 and equal to or less than a value obtained by multiplying a square root of the x by 0.025, and there is no location where the distance is less than a value obtained by multiplying the square root of the x by 0.0025.

The heat transfer member 58 is made of a silicone foaming material having heat resistance capable of withstanding 150° C. or higher, and has thermal conductivity and elasticity. As illustrated in FIG. 7, the heat transfer member 58 is disposed between the outer surfaces of the second support member 562 on both sides in the Y axis direction and the inner surfaces of the rear casing 522 on both sides in the Y axis direction, and is thermally connected to the second support member 562 and the rear casing 522. In the present embodiment, four heat transfer members 58 are provided. The heat transfer member 58 is provided at a position not overlapping the fastening structure 50 when viewed along the Y axis.

The thermally conductive sheet 59 is a sheet made of a material having thermal conductivity, and is made of a graphite sheet in the present embodiment. As illustrated in FIGS. 5 and 8, the thermally conductive sheet 59 includes a first seat portion 591 opened on the optical axis Ax and a pair of second seat portions 592 extending from end edges on both sides of the first seat portion 591 in the Y axis direction toward the proximal end side Ar2. In the thermally conductive sheet 59, on the back surface of the front casing 521, the pair of second seat portions 592 is attached to the back surfaces on both sides in the Y-axis direction, and the first seat portion 591 is attached to the back surface of the distal end portion 5211. That is, the thermally conductive sheet 59 is attached to the back surface of the exterior casing 52 so as to straddle a position overlapping the fastening structure 50 as viewed from the Y axis direction on the back surfaces on both sides of the exterior casing 52 in the Y axis direction and the back surface of the distal end portion 5211 of the front casing 521.

The heat sources in the camera head 5 are the imager 544 and the circuit board 542.

First, the heat of the imager 544 is transferred to the exterior casing 52 following the first heat transfer path of the imager 544 to the first support member 561 to the fastening structure 50 to the front casing 521. In addition, since the thermally conductive sheet 59 is attached to the back surface of the front casing 521, the heat transferred to the fastening structure 50 following the first heat transfer path is diffused toward the distal end side Ar1 of the front casing 521 by the thermally conductive sheet 59.

In addition, the heat of the circuit board 542 is transferred to the exterior casing 52 along the following second to fourth heat transfer paths.

A second heat transfer path is a path from the circuit board 542 to the second support member 562 to the heat sink 57 to the rear casing 522.

A third heat transfer path is a path of the circuit board 542 to the second support member 562 to the heat transfer member 58 to the rear casing 522.

A fourth heat transfer path is a path of the circuit board 542 to the second support member 562 to the first support member 561 to the fastening structure 50 to the front casing 521. In addition, since the thermally conductive sheet 59 is attached to the back surface of the front casing 521, the heat transferred to the fastening structure 50 following the fourth heat transfer path is diffused toward the distal end side Ar1 of the front casing 521 by the thermally conductive sheet 59.

Next, a method of manufacturing the camera head 5 described above will be described.

First, the operator assembles the coupler unit 51, the front casing 521, the imaging unit 54, the support member 56, the heat sink 57, the heat transfer member 58, and the thermally conductive sheet 59 to form an image-capturing unit 100 (first assembling step).

After the first assembling step, the operator inserts the image-capturing unit 100 into the rear casing 522 (second assembling step).

According to the above-described embodiment, the following effects are obtained.

In the camera head 5 according to the present embodiment, the heat sink 57 described above is provided in the exterior casing 52.

Therefore, according to the camera head 5 of the present embodiment, the heat of the imaging unit 54 (circuit board 542) may be transferred to the exterior casing 52 following the above-described second heat transfer path, and heat dissipation of the imaging unit 54 may be improved.

In particular, without the above-described second heat transfer path, the heat of the circuit board 542 is transferred to the portion of the fastening structure 50 following the above-described fourth heat transfer path. Therefore, the portion of the recess 524 locally heats up. Meanwhile, by providing the second heat transfer path, a part of the heat of the circuit board 542 may be transferred to the rear casing 522 following the second heat transfer path, so that it is possible to avoid the portion of the recess 524 from becoming locally hot.

In addition, in the camera head 5 according to the present embodiment, the heat sink 57 is disposed on a side facing the operating unit 53 with the imaging unit 54 interposed therebetween in the exterior casing 52. Therefore, in design, in the exterior casing 52, the heat of the imaging unit 54 (circuit board 542) may be transferred to a portion where the temperature tends to be low, and it is possible to avoid a part from being locally heated.

In addition, in the camera head 5 according to the present embodiment, the space SP is provided between the heat sink 57 and the rear casing 522. Therefore, it is possible to reduce the weight of the heat sink 57 while improving the heat dissipation of the imaging unit 54, and eventually, it is possible to reduce the weight of the camera head 5.

In addition, in the camera head 5 according to the present embodiment, a surface of the heat sink 57 facing the inner surface of the rear casing 522 has a shape following the inner surface. Therefore, heat may be satisfactorily transferred from the heat sink 57 to the rear casing 522, and heat dissipation of the imaging unit 54 (circuit board 542) may be further improved.

Furthermore, in the camera head 5 according to the present embodiment, the heat transfer member 58 described above is provided in the exterior casing 52. In particular, the heat transfer member 58 is provided at a position where the heat transfer member 58 does not overlap the fastening structure 50 when viewed along the Y axis. Therefore, the third heat transfer path described above is provided by the heat transfer member 58, and a part of the heat of the circuit board 542 may be transferred to a position not overlapping the fastening structure 50 in the rear casing 522 when viewed along the Y axis, following the third heat transfer path. Therefore, it is possible to avoid that the portion of the recess 524 locally heats up. Furthermore, in the camera head 5 according to the present embodiment, the heat transfer member 58 is made of a silicone foaming material having heat resistance capable of withstanding 150° C. or higher. Therefore, the heat transfer member 58 may be made of a material that may withstand autoclaving.

In addition, in the camera head 5 according to the present embodiment, the above-described thermally conductive sheet 59 is attached to the inner surface of the exterior casing 52. In particular, the thermally conductive sheet 59 is attached to the back surface of the exterior casing 52 so as to straddle a position overlapping the fastening structure 50 as viewed from the Y axis direction on the back surfaces on both sides of the exterior casing 52 in the Y axis direction and the back surface of the distal end portion 5211 of the front casing 521. Therefore, the heat locally concentrated on the portion of the recess 524 may be diffused to other portions, and the portion of the recess 524 may be avoided from being locally heated.

In addition, in the camera head 5 according to the present embodiment, when the cross-sectional area obtained by cutting the space formed by the support member 56 and the outer surface of the heat sink 57 with the YZ plane is x, there is a location where the distance between the support member 56 or the outer surface of the heat sink 57 and the inner surface of the exterior casing 52 is equal to or more than a value obtained by multiplying the square root of the x by 0.0025 and equal to or less than a value obtained by multiplying the square root of the x by 0.025, and there is no location where the distance is less than the value obtained by multiplying the square root of x by 0.0025. Therefore, it is possible to realize a structure that achieves both heat dissipation of the imaging unit 54 and assemblability of the camera head 5.

Although the embodiments for carrying out the present disclosure have been described so far, the present disclosure should not be limited only by the above-described embodiments.

Configurations of first to third modifications described below may be adopted.

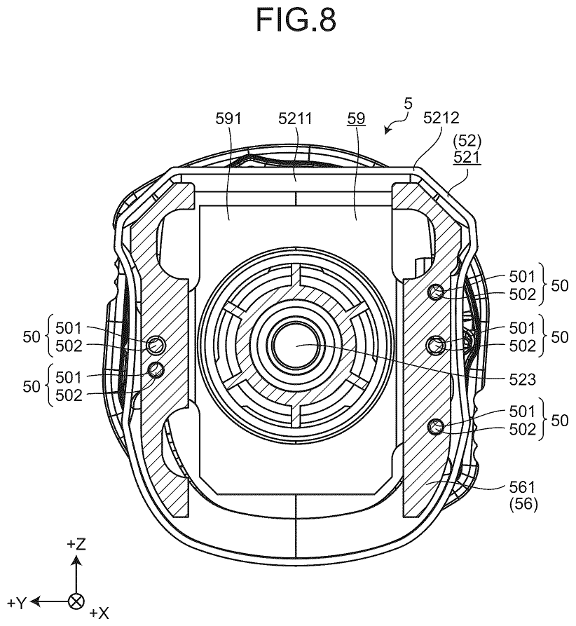

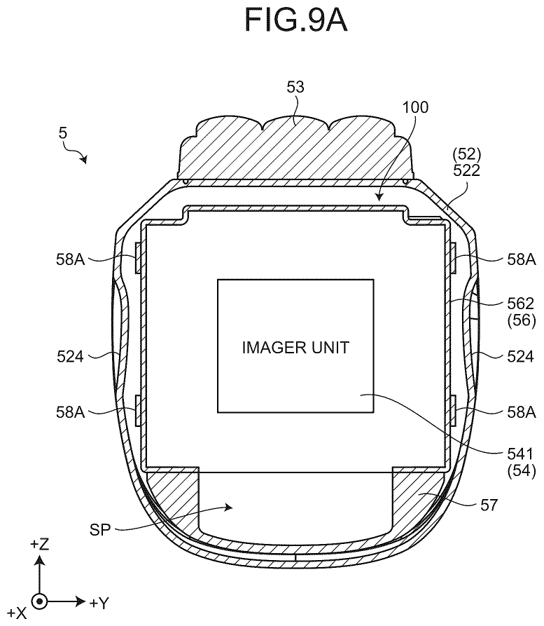

FIGS. 9A and 9B are views illustrating a first modification of the embodiment. Specifically, FIGS. 9A and 9B are cross-sectional views corresponding to FIG. 7. FIG. 9A illustrates a state in which air is not introduced into a heat transfer member 58A. FIG. 9B illustrates a state in which air is introduced into the heat transfer member 58A.

In the above-described embodiment, the heat transfer member 58A according to the first modification illustrated in FIG. 9 may be adopted instead of the heat transfer member 58.

The heat transfer member 58A is formed of, for example, an aluminum sheet, and is a balloon-shaped member that expands when air is introduced into the inside.

Hereinafter, a method of manufacturing the camera head 5 according to the first modification will be described.

First, the operator assembles the coupler unit 51, the front casing 521, the imaging unit 54, the support member 56, the heat sink 57, the heat transfer member 58A, and the thermally conductive sheet 59 (first assembling step). In the first assembling step, as illustrated in FIG. 9A, air is not introduced into the heat transfer member 58A.

After the first assembling step, the operator inserts the image-capturing unit 100 into the rear casing 522 (second assembling step). Also in the second assembling step, as illustrated in FIG. 9A, air is not introduced into the heat transfer member 58A.

After the second assembling step, as illustrated in FIG. 9B, the operator inflates the heat transfer member 58A by introducing air into the heat transfer member 58A, and thermally connects the heat transfer member 58A to the second support member 562 and the rear casing 522 (expansion step). The inlet of the air is not blocked so that the expanded heat transfer member 58A is not expanded more than necessary due to thermal expansion and does not burst.

Even in the case of adopting the heat transfer member 58A as in the first modification described above, the same effects as those of the above-described embodiment are obtained.

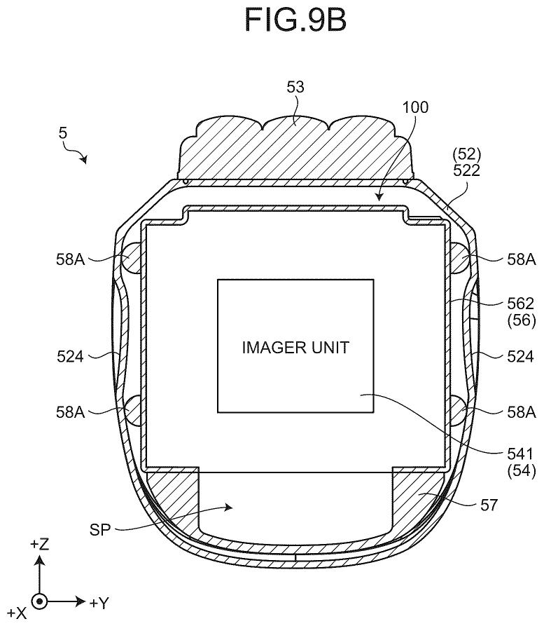

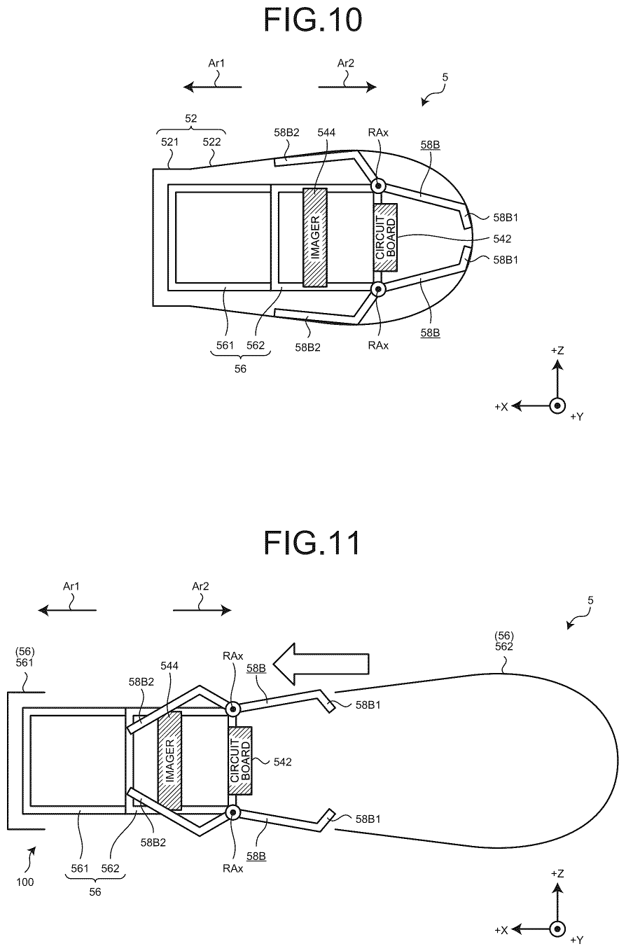

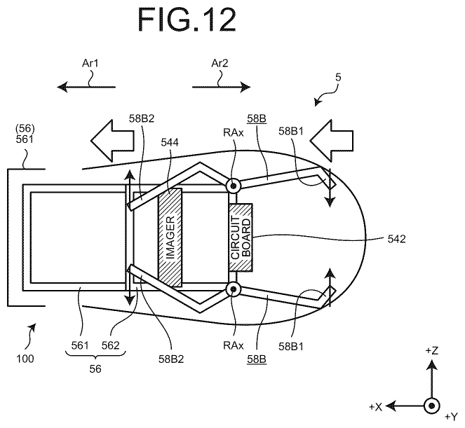

FIGS. 10 to 12 are views for describing a second modification of the embodiment. Specifically, FIG. 10 is a view corresponding to FIG. 4, and is a view illustrating the camera head 5 according to the second modification. FIGS. 11 and 12 are views describing a method of manufacturing the camera head 5 according to the second modification. Note that illustration of the coupler unit 51, the heat sink 57, and the thermally conductive sheet 59 is omitted in FIGS. 10 to 12 for convenience of description.

In the above-described embodiment, a heat transfer member 58B according to the second modification illustrated in FIG. 10 may be adopted instead of the heat transfer member 58.

The heat transfer member 58B is a member made of a thermally conductive material such as metal and extending in the X-axis direction. The heat transfer member 58B is pivotally supported rotatably about a rotation axis RAx (FIG. 10) along the Y axis with respect to the second support member 562 at a substantially central portion in the X axis direction. In the second modification, two heat transfer members 58B are provided so as to face each other along the Z-axis direction.

Hereinafter, a method of manufacturing the camera head 5 according to the second modification will be described.

First, as illustrated in FIG. 11, the operator assembles the coupler unit 51, the front casing 521, the imaging unit 54, the support member 56, the heat sink 57, the heat transfer member 58B, and the thermally conductive sheet 59 to form the image-capturing unit 100 (first assembling step).

After the first assembling step, the operator inserts the image-capturing unit 100 into the rear casing 522 (second assembling step). As a result, as illustrated in FIG. 12, the heat transfer member 58B rotates about the rotation axis RAx while a first portion 58B1 on the proximal end side Ar2 slides with respect to the inner surface of the rear casing 522. In the heat transfer member 58B, a second portion 58B2 located on the opposite side to the first portion 58B1 with the rotation axis Rax interposed therebetween is thermally connected to the inner surface of the rear casing 522.

Even in the case of adopting the heat transfer member 58B as in the second modification described above, the same effects as those of the above-described embodiment are obtained.

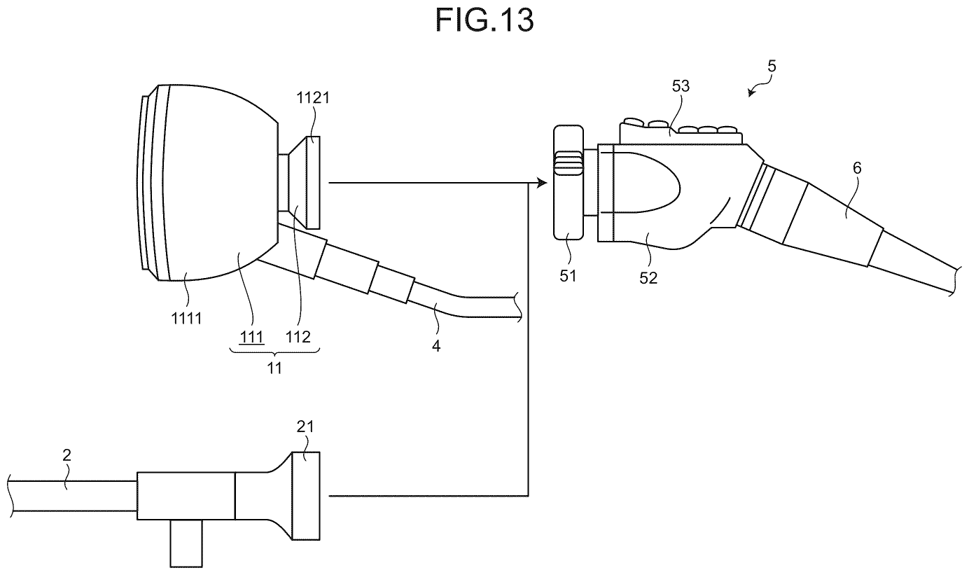

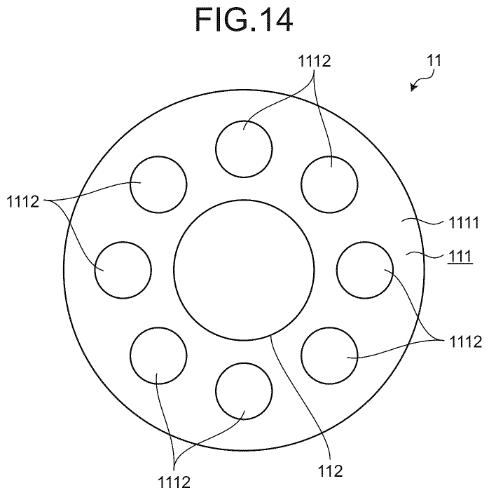

FIGS. 13 and 14 are views for describing a third modification of the embodiment. Specifically, FIG. 13 is a view of an external scope 11 as viewed along the Y axis. FIG. 14 is a view of the external scope 11 as viewed from the front side along the X axis.

In the third modification, in addition to the insertion unit 2 described in the above-described embodiment and the first and second modifications, the external scope 11 illustrated in FIGS. 13 and 14 is detachably connected to the camera head 5. That is, as illustrated in FIG. 13, there are a case where the insertion unit 2 is connected and a case where the external scope 11 is connected to the camera head 5 according to the use state of the user.

The external scope 11 is not inserted into the living body unlike the insertion unit 2, but supplies illumination light to a surgical site and takes in the illumination light (subject image) via the surgical site. As illustrated in FIGS. 13 and 14, the external scope 11 includes an illumination unit 111 and a subject image capturing unit 112 that takes in a subject image.

As illustrated in FIGS. 13 and 14, the illumination unit 111 includes a casing 1111 and a plurality of illumination lenses 1112.

The casing 1111 has an annular shape centered on the optical axis Ax. The other end of the light guide 4 is detachably connected to the casing 1111.

As illustrated in FIG. 14, the plurality of illumination lenses 1112 are disposed at predetermined intervals along the circumferential direction centered on the optical axis Ax on the end surface on the front side of the casing 1111. Then, the plurality of illumination lenses 1112 irradiate the surgical site with the light supplied from the light source device 3 and introduced into the casing 1111 via the light guide 4.

The subject image capturing unit 112 extends along the optical axis Ax. Furthermore, in the subject image capturing unit 112, there is provided an optical system that includes one or a plurality of lenses and collects the subject image emitted from the plurality of illumination lenses 1112 and passing through the surgical site.

Furthermore, a connecting unit 1121 is provided at an end portion of the subject image capturing unit 112 on the proximal end side Ar2. The connecting unit 1121 is designed (shaped) to be compatible with the eyepiece unit 21 in the insertion unit 2, and is detachably connected to the coupler unit 51. For example, an outer diameter shape of the connecting unit 1121 is defined in ISO/TS 18339:2015, and by using the external scope 11 having the defined shape, compatible connection with the camera head 5 becomes possible. Note that, optically, the eyepiece unit 21 having a general eyepiece diopter of −1 to 0^{m-1} is preferable in terms of versatility, but the present disclosure is not necessarily limited thereto as long as it is within the focus range of the camera head 5 to be combined.

According to the third modification described above, the following effects are obtained in addition to the same effects as those of the above-described embodiment.

The above-described external scope 11 is detachably connected to the camera head 5 according to the third modification. Therefore, in addition to observing the inside of the living body by inserting the insertion unit 2 into the living body at the time of endoscopic surgery, it is possible to enlarge and observe the surgical site using the external scope 11 at the time of laparotomy. That is, convenience may be improved.

Note that the following configurations also belong to the technical scope of the present disclosure.

(1) A camera head including: an exterior casing configured to be connected to an insertion unit, the insertion unit being inserted into a subject and configured to take in a subject image from the subject; an imaging unit provided in the exterior casing and configured to capture the subject image; and a heat sink provided in the exterior casing and configured to transfer heat generated in the imaging unit to the exterior casing.

(2) The camera head according to (1), further including an operating unit provided on an outer surface of the exterior casing and including a button configured to receive an operation by a user, wherein the heat sink is disposed on a side facing the operating unit with the imaging unit interposed therebetween in the exterior casing.

(3) The camera head according to (1) or (2), further including a support member provided in the exterior casing and supports the imaging unit,
wherein the heat sink is thermally connected to the support member and configured to transfer heat generated in the imaging unit and transferred via the support member to the exterior casing.

(4) The camera head according to any one of (1) to (3), wherein a space is provided between the heat sink and the support member.

(5) The camera head according to any one of (1) to (4), wherein a surface of the heat sink facing an inner surface of the exterior casing has a shape following an inner surface of the exterior casing.

(6) The camera head according to any one of (1) to (5), further including: a support member provided in the exterior casing and supports the imaging unit; and a heat transfer member thermally connected to each of the support member and an inner surface of the exterior casing and configured to transfer heat generated in the imaging unit and transferred via the support member to the exterior casing.

(7) The camera head according to (6), wherein the heat transfer member is thermally connected to each of the support member and an inner surface of the exterior casing at a position other than a fastening unit where the support member and the exterior casing are fastened.

(8) The camera head according to (7), wherein a recess that is recessed toward the inside and gripped by a user is provided on an outer surface of the exterior casing, and the fastening unit is disposed to face a back surface of the recess.

(9) The camera head according to any one of (6) to (8), wherein the heat transfer member is made of a material having heat resistance capable of withstanding 150° C. or higher.

(10) The camera head according to any one of (6) to (8), wherein the heat transfer member is made of a silicone-based foam material.

(11) The camera head according to any one of (1) to (10), wherein a thermally conductive member configured to diffuse heat transferred to the exterior casing is provided on an inner surface of the exterior casing.

(12) The camera head according to (11), further including a support member provided in the exterior casing and configured to support the imaging unit, wherein the thermally conductive member is attached so as to straddle between a position of a fastening unit where the support member and the exterior casing are fastened and a position separated from the fastening unit.

(13) The camera head according to (11), wherein the thermally conductive member is disposed on each of: back surfaces of two surfaces positioned between an arrangement surface of an operating unit having a button for receiving an operation by a user in the exterior casing and a surface facing the arrangement surface of the operating unit; and a back surface of a distal end portion of the exterior casing.

(14) The camera head according to (12), wherein a recess that is recessed toward the inside and gripped by a user is provided on an outer surface of the exterior casing, and the fastening unit is disposed to face a back surface of the recess.

(15) The camera head according to any one of (1) to (14), further including a support member provided in the exterior casing and supports the imaging unit, wherein where a cross-sectional area obtained by cutting a space formed by the support member and an outer surface of the heat sink by a plane orthogonal to an optical axis of the imaging unit is x, there is a location where a distance between the support member or the outer surface of the heat sink and an inner surface of the exterior casing is equal to or more than a value obtained by multiplying a square root of the x by 0.0025 and equal to or less than a value obtained by multiplying the square root of the x by 0.025, and there is no location where the distance is less than a value obtained by multiplying the square root of the x by 0.0025.

(16) An endoscope system including: an insertion unit configured to be inserted into a subject and take in a subject image from the subject; a camera head configured to be connected to the insertion unit and capture the subject image; and a control device configured to process an image captured by the camera head, wherein the camera head includes an exterior casing configured to be connected to the insertion unit, an imaging unit provided in the exterior casing and configured to capture the subject image, and a heat sink provided in the exterior casing and configured to transfer heat generated in the imaging unit to the exterior casing.

(17) A method of manufacturing a camera head including: an exterior casing configured to be connected to an insertion unit, the insertion unit being inserted into a subject and configured to take in a subject image from the subject; an imaging unit provided in the exterior casing and configured to capture the subject image; and a heat sink provided in the exterior casing and configured to transfer heat generated in the imaging unit to the exterior casing, the method including: a first assembling step of assembling the imaging unit and the heat sink to form an image-capturing unit; and a second assembling step of inserting the image-capturing unit into the exterior casing and assembling the image-capturing unit and the exterior casing after the first assembling step.

(18) The method according to (17), wherein the camera head includes a support member provided in the exterior casing and supports the imaging unit, and a heat transfer member thermally connected to the support member and an inner surface of the exterior casing, and transfers heat generated in the imaging unit and transferred via the support member to the exterior casing, the heat sink is thermally connected to the support member, and transfers heat generated in the imaging unit and transferred via the support member to the exterior casing, the heat transfer member expands when air is introduced, and is thermally connected to the support member and an inner surface of the exterior casing, in the first assembling step, the imaging unit, the support member, the heat sink, and the heat transfer member are assembled to form the image-capturing unit, and the method further includes an expansion step of expanding the heat transfer member by introducing air into the heat transfer member after the second assembling step, and thermally connecting the heat transfer member to the support member and the inner surface of the exterior casing.

(19) The method according to (17), wherein the camera head includes a support member provided in the exterior casing and configured to support the imaging unit, and a heat transfer member thermally connected to the support member and an inner surface of the exterior casing and configured to transfer heat generated in the imaging unit and transferred via the support member to the exterior casing, the heat sink is thermally connected to the support member, and transfers heat generated in the imaging unit and transferred via the support member to the exterior casing, the heat transfer member is rotatably and pivotally supported with respect to the support member, in the first assembling step, the imaging unit, the support member, the heat sink, and the heat transfer member are assembled to form the image-capturing unit, and in the second assembling step, the image-capturing unit is inserted into the exterior casing, the heat transfer member is rotated while a first portion of the heat transfer member is slid with respect to an inner surface of the exterior casing, and the image-capturing unit and the exterior casing are assembled in a state where a second portion located on a side opposite to the first portion with a rotation point of the heat transfer member pivotally supported with respect to the support member interposed therebetween is thermally connected to the inner surface of the exterior casing.

According to the camera head, the endoscope system, and the method of manufacturing the camera head of the present disclosure, it is possible to improve heat dissipation of heat generated in the imaging unit.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A camera head comprising:
an exterior casing configured to be connected to an insertion unit, the insertion unit being inserted into a subject and configured to take in a subject image from the subject;
an imaging unit provided in the exterior casing and configured to capture the subject image; and
a heat sink provided in the exterior casing and configured to transfer heat generated in the imaging unit to the exterior casing.

2. The camera head according to claim 1, further comprising an operating unit provided on an outer surface of the exterior casing and including a button configured to receive an operation by a user,
wherein the heat sink is disposed on a side facing the operating unit with the imaging unit interposed therebetween in the exterior casing.

3. The camera head according to claim 1, further comprising a support member provided in the exterior casing and supports the imaging unit,
wherein the heat sink is thermally connected to the support member and configured to transfer heat generated in the imaging unit and transferred via the support member to the exterior casing.

4. The camera head according to claim 3, wherein a space is provided between the heat sink and the support member.

5. The camera head according to claim 1, wherein a surface of the heat sink facing an inner surface of the exterior casing has a shape following an inner surface of the exterior casing.

6. The camera head according to claim 1, further comprising:
a support member provided in the exterior casing and supports the imaging unit; and
a heat transfer member thermally connected to each of the support member and an inner surface of the exterior casing and configured to transfer heat generated in the imaging unit and transferred via the support member to the exterior casing.

7. The camera head according to claim 6, wherein the heat transfer member is thermally connected to each of the support member and an inner surface of the exterior casing at a position other than a fastening unit where the support member and the exterior casing are fastened.

8. The camera head according to claim 7, wherein
a recess that is recessed toward the inside and gripped by a user is provided on an outer surface of the exterior casing, and
the fastening unit is disposed to face a back surface of the recess.

9. The camera head according to claim 6, wherein the heat transfer member is made of a material having heat resistance capable of withstanding 150° C. or higher.

10. The camera head according to claim 6, wherein the heat transfer member is made of a silicone-based foam material.

11. The camera head according to claim 1, wherein a thermally conductive member configured to diffuse heat transferred to the exterior casing is provided on an inner surface of the exterior casing.

12. The camera head according to claim 11, further comprising a support member provided in the exterior casing and configured to support the imaging unit,
wherein the thermally conductive member is attached so as to straddle between a position of a fastening unit where the support member and the exterior casing are fastened and a position separated from the fastening unit.

13. The camera head according to claim 11, wherein the thermally conductive member is disposed on each of: back surfaces of two surfaces positioned between an arrangement surface of an operating unit having a button for receiving an operation by a user in the exterior casing and a surface facing the arrangement surface of the operating unit; and a back surface of a distal end portion of the exterior casing.

14. The camera head according to claim 12, wherein
a recess that is recessed toward the inside and gripped by a user is provided on an outer surface of the exterior casing, and
the fastening unit is disposed to face a back surface of the recess.

15. The camera head according to claim 1, further comprising a support member provided in the exterior casing and supports the imaging unit,
wherein where a cross-sectional area obtained by cutting a space formed by the support member and an outer surface of the heat sink by a plane orthogonal to an optical axis of the imaging unit is x,
there is a location where a distance between the support member or the outer surface of the heat sink and an inner surface of the exterior casing is equal to or more than a value obtained by multiplying a square root of the x by 0.0025 and equal to or less than a value obtained by multiplying the square root of the x by 0.025, and there is no location where the distance is less than a value obtained by multiplying the square root of the x by 0.0025.

16. An endoscope system comprising:
an insertion unit configured to be inserted into a subject and take in a subject image from the subject;
a camera head configured to be connected to the insertion unit and capture the subject image; and
a control device configured to process an image captured by the camera head,
wherein the camera head includes
an exterior casing configured to be connected to the insertion unit,
an imaging unit provided in the exterior casing and configured to capture the subject image, and
a heat sink provided in the exterior casing and configured to transfer heat generated in the imaging unit to the exterior casing.

17. A method of manufacturing a camera head including: an exterior casing configured to be connected to an insertion unit, the insertion unit being inserted into a subject and configured to take in a subject image from the subject; an imaging unit provided in the exterior casing and configured to capture the subject image; and a heat sink provided in the exterior casing and configured to transfer heat generated in the imaging unit to the exterior casing, the method comprising:
a first assembling step of assembling the imaging unit and the heat sink to form an image-capturing unit; and
a second assembling step of inserting the image-capturing unit into the exterior casing and assembling the image-capturing unit and the exterior casing after the first assembling step.

18. The method according to claim 17, wherein
the camera head includes
a support member provided in the exterior casing and supports the imaging unit, and
a heat transfer member thermally connected to the support member and an inner surface of the exterior casing, and transfers heat generated in the imaging unit and transferred via the support member to the exterior casing,
the heat sink is thermally connected to the support member, and transfers heat generated in the imaging unit and transferred via the support member to the exterior casing,
the heat transfer member expands when air is introduced, and is thermally connected to the support member and an inner surface of the exterior casing,
in the first assembling step, the imaging unit, the support member, the heat sink, and the heat transfer member are assembled to form the image-capturing unit, and
the method further comprises an expansion step of expanding the heat transfer member by introducing air into the heat transfer member after the second assembling step, and thermally connecting the heat transfer member to the support member and the inner surface of the exterior casing.

19. The method according to claim 17, wherein
the camera head includes
a support member provided in the exterior casing and configured to support the imaging unit, and
a heat transfer member thermally connected to the support member and an inner surface of the exterior casing and configured to transfer heat generated in the imaging unit and transferred via the support member to the exterior casing,
the heat sink is thermally connected to the support member, and transfers heat generated in the imaging unit and transferred via the support member to the exterior casing,
the heat transfer member is rotatably and pivotally supported with respect to the support member,
in the first assembling step, the imaging unit, the support member, the heat sink, and the heat transfer member are assembled to form the image-capturing unit, and
in the second assembling step, the image-capturing unit is inserted into the exterior casing, the heat transfer member is rotated while a first portion of the heat transfer member is slid with respect to an inner surface of the exterior casing, and the image-capturing unit and the exterior casing are assembled in a state where a second portion located on a side opposite to the first portion with a rotation point of the heat transfer member pivotally supported with respect to the support member interposed therebetween is thermally connected to the inner surface of the exterior casing.
